(19) United States
(12) Patent Application Publication
(10) Pub. No.: US 2020/0397617 A1
(43) Pub. Date: Dec. 24, 2020

(54) SYSTEMS, APPARATUS, AND METHODS FOR DELIVERY OF THERAPEUTIC SUBSTANCE TO THE TYMPANIC MEMBRANE

(71) Applicant: TUSKER MEDICAL, INC., Menlo Park, CA (US)

(72) Inventors: Eric GOLDFARB, Belmont, CA (US); Rohit GIROTRA, San Francisco, CA (US)

(73) Assignee: TUSKER MEDICAL, INC., Menlo Park, CA (US)

(21) Appl. No.: 17/007,560

(22) Filed: Aug. 31, 2020

Related U.S. Application Data
(62) Division of application No. 15/892,033, filed on Feb. 8, 2018.
(60) Provisional application No. 62/456,342, filed on Feb. 8, 2017.

(51) Int. Cl. A61F 11/00; A61M 31/00; A61F 13/20; A61F 13/537; A61F 13/12; A61K 9/00; A61K 9/06; A61K 47/34; A61F 13/36

(52) U.S. Cl. CPC A61F 11/00; A61M 31/002; A61F 13/2017; A61F 13/537; A61F 13/2074; A61F 2013/530802; A61K 9/0046; A61K 9/06; A61K 47/34; A61F 13/36; A61F 13/12

(57) ABSTRACT

Systems, apparatus, and methods are described for delivering a therapeutic substance to the tympanic membrane of an ear of a subject, including a reservoir configured to contain the therapeutic substance and a delivery interface by which the therapeutic substance is delivered to the tympanic membrane.

### CROSS-REFERENCE TO RELATED APPLICATIONS

This application is a divisional application of U.S. patent application Ser. No. 15/892,033, filed Feb. 8, 2018, and titled “SYSTEMS, APPARATUS, AND METHODS FOR DELIVERY OF THERAPEUTIC SUBSTANCE TO THE TYMPANIC MEMBRANE,” which claims priority to and the benefit of U.S. Provisional Patent Application No. 62/456,342, filed Feb. 8, 2017 and titled “SYSTEMS, APPARATUS, AND METHODS FOR DELIVERY OF THERAPEUTIC SUBSTANCE TO THE TYMPANIC MEMBRANE.” Both applications are incorporated herein by reference as if reproduced in full below.

### TECHNICAL FIELD

The present disclosure relates generally to systems, apparatus, and methods for delivering a substance to an ear of a subject. More specifically, the present disclosure relates to systems, apparatus, and methods for accessing and delivering a therapeutic substance, such as an anesthetic, to the subject's tympanic membrane.

### BACKGROUND

In humans and many other animals, the tympanic cavity is a small cavity in the petrous temporal bone. As shown in FIG. 1, the tympanic cavity surrounds the bones of the middle ear, including the malleus, incus, and stapes, and is bounded by six walls. The convex lateral wall, which separates the tympanic cavity from the external auditory canal and outer ear, includes the tympanic membrane. The convex medial wall, which separates the tympanic cavity from the inner ear, includes the oval window, round window, and the promontory formed by the first turn of the cochlea. The roof separates the tympanic cavity from the cranial cavity, the floor separates the tympanic cavity from the jugular vein, the posterior wall separates the tympanic cavity from the mastoid antrum air cells, and the anterior wall separates the tympanic cavity from the carotid canal.

The tympanic cavity is hollow and normally filled with air; however, the air of the tympanic cavity usually is not in direct contact with the atmosphere of the outside environment. When a pressure difference develops between the tympanic cavity and the outside environment (e.g., because the subject moves between altitudes as on an airplane or dives into water, the tympanic membrane may become damaged if it is not relieved. If tympanic cavity pressure remains lower than the atmosphere, the tympanic membrane may retract into the tympanic cavity. If tympanic cavity pressure remains higher than the atmosphere, the tympanic membrane may rupture.

Otitis media is an inflammation of the middle ear (without reference to etiology or pathogenesis) and is particularly common in human children due to their anatomy and immune function. If severe or untreated, otitis media may result in rupture of the tympanic membrane, hearing loss, or intracranial complications. Otitis media may be managed using oral and topical pain killers (e.g., ibuprofen, acetaminophen, opiates, antipyrine, benzocaine ear drops) and antibiotics (e.g., amoxicillin, amoxicillin-clavulanate, beta lactamase inhibitor).

The Eustachian tube helps equalize middle ear pressure and drain fluid from the middle ear by connecting the tympanic cavity to the nasopharynx; however, Eustachian tubes are susceptible to inflammation and dysfunction.

To relieve pressure or pain from pressure differences and/or inflammation, the tympanic membrane may be punctured (e.g., by performing a myringotomy, tympanostomy, or tympanocentesis). Fluid present in the middle ear may be aspirated during a procedure. In some procedures, a tube or grommet is inserted into the tympanic membrane to drain fluid from the tympanic cavity. The tympanic membrane must be anesthetized before it is punctured, to avoid inflicting pain on the subject during the procedure.

The footplate of the stapes in the middle ear connects to the oval window, a membrane-covered opening that leads from the middle ear to the inner ear, which is a labyrinth of fluid-filled tubes and sacs, including the vestibule, semicircular canals, and cochlea. When the stapes presses on the oval window, the fluid (e.g., perilymph) of the inner ear moves, thus converting energy from mechanical vibrations to waves in the fluid and detecting hair cells of the inner ear. The round window is a second opening between the middle and inner ear covered by a membrane, which vibrates with opposite phase to vibrations entering the inner ear through the oval window. The cochlea transmits sound signals to the brain, while the vestibular system (i.e., the vestibule and semicircular canals) provide the brain with information about head turns, translations, and tilts used for balance, spatial orientation, and motion.

Vestibular neuritis and labyrinthitis are inflammations of the inner ear (without reference to etiology or pathogenesis) caused by viral infections, bacterial infections, autoimmune disorders, and/or physical blockage of the inner ear resulting in symptoms that include dizziness, vertigo, nausea, tinnitus, and hearing changes. Neuritis and labyrinthitis may be managed using oral and topical (via, e.g., transtympanic perfusion and cochlear microperfusion) antioxidants, antiviral agents, anti-inflammatories, immunosuppressive agents, and antibiotics (e.g., broad-spectrum antibiotics or combination therapy with central nervous system penetration).

In addition to inflammation and infection, both the middle and inner ear are susceptible to other disorders including, but not limited to, otosclerosis or otospongiosis (abnormal bone growth near the middle ear), which may be managed using sodium fluoride or bisphosphonates; and Ménière's disease, which may be treated with, for example, gentamicin as part of a chemical labyrinthectomy.

### SUMMARY

Systems, apparatus, and methods are described for delivering a therapeutic substance to an ear of a subject, in particular to the tympanic membrane of the subject. The therapeutic substance may be an anesthetic, such as lidocaine, which may be used to anesthetize the tympanic membrane in preparation for piercing the membrane, e.g., for a myringotomy or tympanostomy, or tympanocentesis. The delivery system and method can enable delivery of the therapeutic substance over a sufficient time to deliver a therapeutically effective dose, without requiring the subject to be still and/or to maintain an awkward head position or orientation, e.g., with their head tilted to the side to allow drops of liquid therapeutic substance to be maintained in position against the tympanic membrane. The therapeutic substance is also preferably delivered without allowing it to pass through an existing perforation in the tympanic membrane into the middle ear. The delivery system preferably provides soft, non-painful contact with the tympanic membrane and/or ear canal.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Other systems, processes, and features will become apparent to those skilled in the art upon examination of the following drawings and detailed description. It is intended that all such additional systems, processes, and features be included within this description, be within the scope of the present invention, and be protected by the accompanying claims.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 illustrates the anatomy of the human ear.

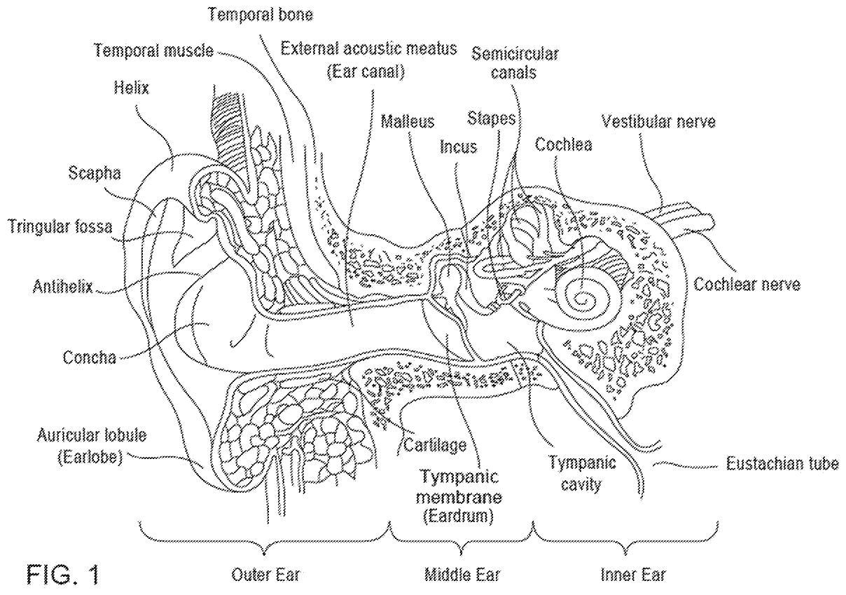

FIG. 2 is a schematic illustration of a therapeutic delivery system according to an embodiment.

FIG. 3 is a flow diagram illustrating a method for delivering a therapeutic substance to a tympanic membrane of a subject in accordance with some embodiments.

FIGS. 4A-4B are schematic illustrations of a therapeutic substance delivery system according to an embodiment.

FIGS. 5A-5B are schematic illustrations of a therapeutic substance delivery system according to an embodiment.

FIGS. 6A-6B are schematic illustrations of a therapeutic substance delivery system according to an embodiment.

FIGS. 7A-7B are schematic illustrations of a therapeutic substance delivery system according to an embodiment.

FIGS. 8A-8B are schematic illustrations of a therapeutic substance delivery system according to an embodiment.

### DETAILED DESCRIPTION

Systems, apparatus, and methods are described herein for delivery of therapeutic substances to the middle ear, and in particular to the tympanic cavity. In some embodiments, an apparatus can include a fibrous wicking body having a proximal end and a distal end. The fibrous wicking body can be configured to conduct a fluid including a therapeutic substance from the proximal end to the distal end. The distal end can include a delivery interface configured to be placed in operative apposition with a tympanic membrane such that the delivery interface can deliver the therapeutic substance to the tympanic membrane.

In some embodiments, a method can include disposing a delivery system including a delivery interface in an ear canal of an ear such that the delivery interface is in operative apposition with a tympanic membrane of the ear. The delivery system can be configured to change configuration after being disposed in the ear canal. The delivery interface can be maintained in operative apposition with the tympanic membrane until a therapeutically effective dose of the therapeutic substance is delivered, via the delivery interface, to the tympanic membrane.

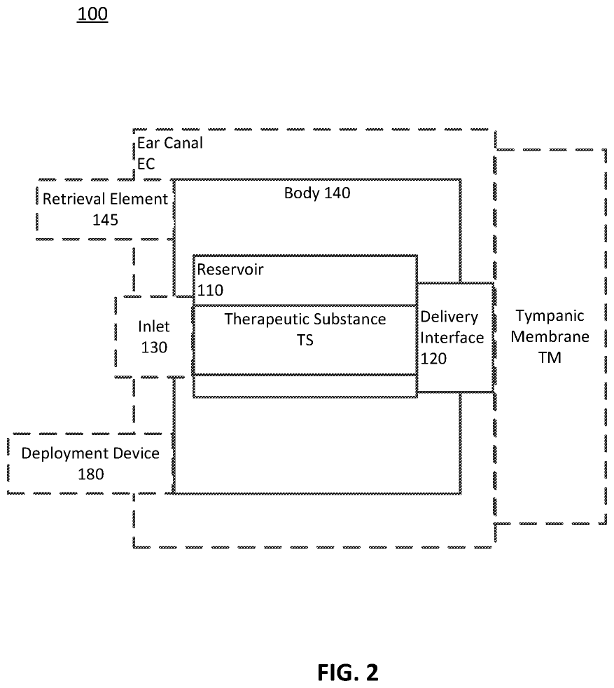

As illustrated schematically in FIG. 2, a delivery system 100 can include a reservoir 110 that can contain a therapeutic substance TS. A delivery interface 120 can be part of, or coupled fluidically to, reservoir 110, enabling therapeutic substance TS to contact, and enter, the tympanic membrane TM. Optionally, an inlet 130 can be coupled fluidically to reservoir 110, enabling reservoir 110 to be supplied (e.g., filled or refilled), with therapeutic substance TS. Delivery system 100 can include a body or housing 140 that supports, or defines, reservoir 110, delivery interface 120, and/or inlet 130. Delivery system 100 may also include a retrieval element 145 by which system 100 can be removed from the ear canal EC of the subject. Delivery system 100 may be deployed into an operative position in the ear canal EC of the subject with a deployment device 180, which may be releasably engaged with delivery system 100 in preparation for deployment of delivery system 100, and released from delivery system 100 after deployment in ear canal EC.

Each component of delivery system 100 can be implemented in various ways. Reservoir 110 may be formed by a solid structure within which therapeutic substance TS (in fluid and/or solid form) may be eluted, diffused, or released by other mechanisms. For example, reservoir 110 may be an open cell foam in which therapeutic substance TS in liquid form is contained and from which therapeutic substance TS may be released by osmotic diffusion from a distal surface of the foam reservoir that is in contact with the tympanic membrane TM (the delivery interface 120). That is, delivery or release of therapeutic substance TS may be driven by a differential in concentration of therapeutic substance TS in the foam and in the tympanic membrane TM. The foam reservoir 110 may be delivered by mechanical insertion through the ear canal EC into operative apposition with the tympanic membrane TM. As needed, therapeutic substance TS may be added to reservoir 110 by supplying it, e.g., in liquid form, to a proximal surface of the foam, serving as inlet 130.

In another embodiment, reservoir 110 may be in the form of a solid structure that degrades (e.g., biodegrades). In some embodiments, reservoir 110 can release therapeutic substance TS as reservoir 110 degrades. In some embodiments, reservoir 110 may be in the form of a solid structure that biodegrades, i.e. formed of a material that breaks down in contact with tissue, and can release therapeutic substance TS as reservoir 110 biodegrades.

In other embodiments, reservoir 110 may be formed of a carrier material in which therapeutic substance TS in liquid form is mixed, or in solid, particulate form is dissolved, suspended, etc. The carrier material and therapeutic substance TC may be delivered through the ear canal EC into operative apposition with the tympanic membrane, for example by injection, spray, or other suitable delivery mechanism. Thus, reservoir 110 may be formed in situ on tympanic membrane TM. The carrier material and therapeutic substance may be delivered as a foam or mousse or a hydrogel. In another embodiment, the carrier may be deliverable in a liquid form that changes to a solid form, e.g. by a change in temperature. Such carriers are known from applications such as liquid bandages. In this embodiment, the carrier, containing therapeutic substance TC, may be injected or sprayed in liquid form against the surface of tympanic membrane TM and then solidify due to the higher temperature of the tympanic membrane, thus forming reservoir 110 in situ on tympanic membrane TM.

In another embodiment, reservoir 110 may be in the form of a stable/solid thin film that may contain therapeutic substance TS as with the other embodiments described above, and the therapeutic substance TS may be delivered to tympanic membrane TM by diffusion when the thin film is in apposition with the tympanic membrane TM. Alternatively, the thin film may function as a body 140 on which a reservoir 110 in the form of a foam, hydrogel or other material containing therapeutic substance TS may be carried. In other words, the body 140 may be a thin film substrate, and reservoir 110 may be a layer of material disposed on the distal surface of the body 140.

In another embodiment, reservoir 110 may be in the form of a bundle or other mass of wicking, fibrous material, i.e. a material that can absorb therapeutic substance TS in liquid form, and conduct therapeutic substance TS by capillary action through the body of reservoir 110 to a distal surface of reservoir 110 in contact with tympanic membrane TM, from which the therapeutic substance TS can diffuse into tympanic membrane TM. As needed, therapeutic substance TS may be added to reservoir 110 by supplying it, e.g., in liquid form, to a proximal surface or end of the wicking material, serving as inlet 130.

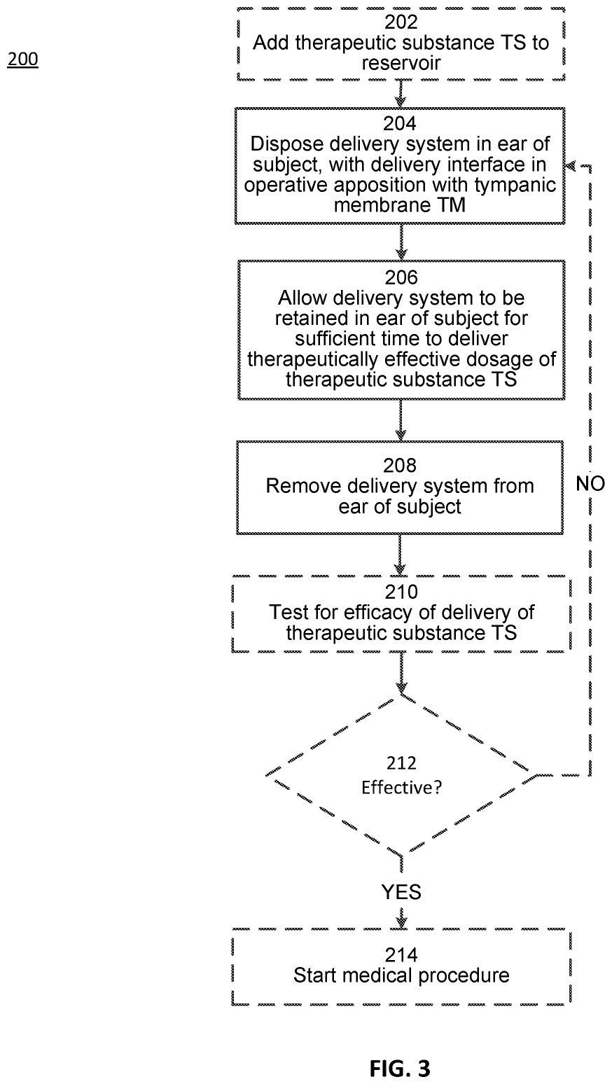

A method of delivering therapeutic substance TS to tympanic membrane TM is illustrated schematically in FIG. 3.

Initially, therapeutic substance TS may be added to the reservoir, such as reservoir 110 described above, in step 202. In step 204, a delivery system, such as delivery system 100 disclosed above, is deployed to the ear of the subject. In particular, deployment includes disposing the delivery system with its delivery interface, such as delivery interface 120, in operative apposition with the tympanic membrane TM of the subject. In some embodiments, the delivery system can change configuration after being disposed in the ear canal. In step 206, the delivery system is maintained (e.g., allowed to be retained) in the ear of the subject for sufficient time to deliver a therapeutically effective dosage of the therapeutic substance TS to the tympanic membrane TM of the subject. In step 208, the delivery system is removed from the ear of the subject.

Optionally, in step 210, a test may be conducted to evaluate the efficacy of the delivery of therapeutic substance TS. For example, if therapeutic substance TS is an anesthetic, the therapeutically effective dosage to be delivered to the tympanic membrane TM of the subject may be the dosage required to anesthetize the tympanic membrane TM so that a medical procedure may performed, such as delivering a tympanostomy tube through an incision formed in the tympanic membrane TM. The efficacy of delivery of the therapeutic substance TM may thus be a test of anesthetization of the membrane, e.g., by touching the membrane and assessing the subject's response. In step 212, if it is determined that the delivery was not effective, the method may revert to step 204 to dispose the, or another, delivery system in the ear to deliver more therapeutic substance TS. Alternatively, if it is determined that the delivery was effective, the method may proceed to step 214, in which a medical procedure, such as delivery of a tympanostomy tube, may be started.

Therapeutic substance TS can be any suitable substance or combination of substances, in any suitable dosage form or combination of dosage forms. Non-limiting examples include analgesics (e.g., non-steroidal anti-inflammatory drugs (NSAIDs) like acetaminophen, COX-2 inhibitors, opioids, flupirtine, cannabinoids, capsaicinoids, etc.), anesthetics (e.g., lidocaine, benzocaine, procaine, amethocaine, cocaine, tetracaine, prilocaine, bupivicaine, levobupivacaine, ropivacaine, mepivacaine, dibucaine, etidocaine, etc.), anti-inflammatories (e.g., NSAIDs like aspirin, ibuprofen, and naproxen, peptides, steroids or glucocorticosteroids like dexamethasone, etc.), antibiotics (e.g., ciprofloxacin, ciprofloxacin otic suspension, amoxicillin, amoxicillin-clavulanate, beta lactamase inhibitor, etc.), antivirals, antifungals, antiparasitics, decongestants (e.g., ephedrine, levomethamphetamine, naphazoline, oxymetazoline, phenylephrine, phenylpropanolamine, propylhexedrine, synephrine, tetrahydrozoline, xylometazoline, pseudoephedrine, tramazoline, etc.), mucokinetics (e.g., mucolytics like acetylcysteine, expectorants like guaifenesin, surfactants, etc.), antihistamines, antioxidants, immunosuppressive agents, and dissociatives (e.g., NMDA receptor antagonists like gacyclidine, K-opioid receptor agonists, etc.). Suitable dosage form(s) can include liquids (including solutions, suspensions, and colloids) delivered (e.g., sprayed) as a liquid, liquid aerosol, foam, emulsion, sol, etc.; gases (including solutions, suspensions, and colloids) delivered as a vapor; and solids (including solutions, suspensions, and colloids) delivered as solid aerosols, solid foam, gel, sol, etc., including solids that are incorporated into or onto a solid or porous substrate for elution or release by biodegradation of the substrate.

Suitable biodegradable solids may include, but are not limited to, agro-polymers, including polysaccharides and proteins, or biopolyesters including natural monomers (e.g., polyhydroxyalkanoates (PHAs) like poly-3-hydroxybutyrate (PHB), polyhydroxyvalerate (PHV) and polyhydroxyhexanoate (PHH), etc.), renewable monomers (e.g., polylactic acid (PLA)), and synthetic monomers (e.g., polybutylene succinate (PBS), polycaprolactone (PCL), etc.). Additional examples of biodegradable solids include polyglycolic acid, poly(lactic-co-glycolic) acid, poly-ε-caprolactone, polydioxanone, chitosan, hyaluronic acid, poly(2-hydroxyethyl-methacrylate), poly(ethylene glycol), polyurethanes, poly(ester amide)s, polyanhydrides, polyvinyl alcohol, cellulose esters, polyethylene terephthalate, and hydrogels. In some embodiments, a normal plastic polymer such as polyethylene or polypropylene may be incorporated with an additive which causes degradation (e.g., due to oxidation). Biodegradable materials may include films, fibers, extruded or molded products, laminates, foams, powders, nonwovens, adhesives, and/or coatings.

Various non-limiting, exemplary embodiments are described below.

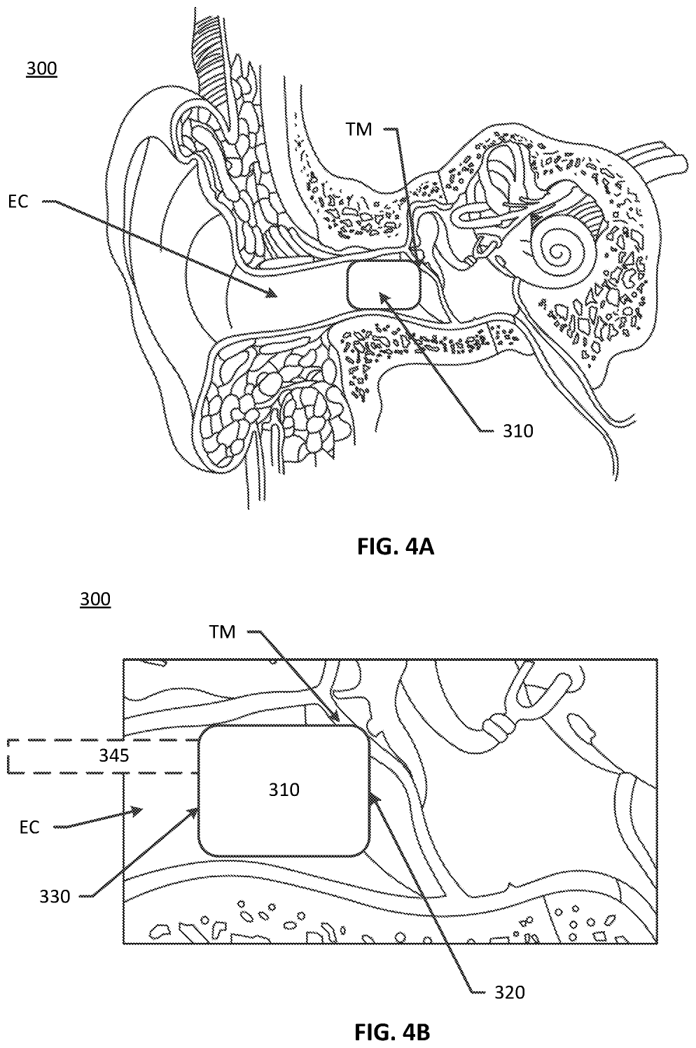

FIGS. 4A and 4B schematically illustrate a delivery system 300 according to a first embodiment. In this embodiment, delivery system 300 includes a reservoir 310 implemented as a unitary solid structure formed of a foam material. Therapeutic substance TS is disposed within the foam material of reservoir 310, and can move through the foam material to delivery interface 320, which is the distal surface of the foam structure.

In use, delivery interface 320 is placed in apposition with tympanic membrane TM, as best seen in FIG. 4B, to permit therapeutic substance TS to be absorbed into tympanic membrane TM, such as by osmotic transport. Reservoir 310 can be supplied (e.g., filled and/or refilled), with therapeutic substance TS, via inlet 330, which is the proximal surface of the foam structure.

Delivery system 300 can be deployed by inserting the foam structure through ear canal EC until delivery interface 320 is in apposition with tympanic membrane TM. Optionally, delivery system 300 can include a retrieval element 345, coupled to reservoir 310, by which a user can remove delivery system 300 from the ear of the subject by pulling delivery system 300 through ear canal EC. The retrieval element 345 can be, for example, a string or a flexible or rigid tab.

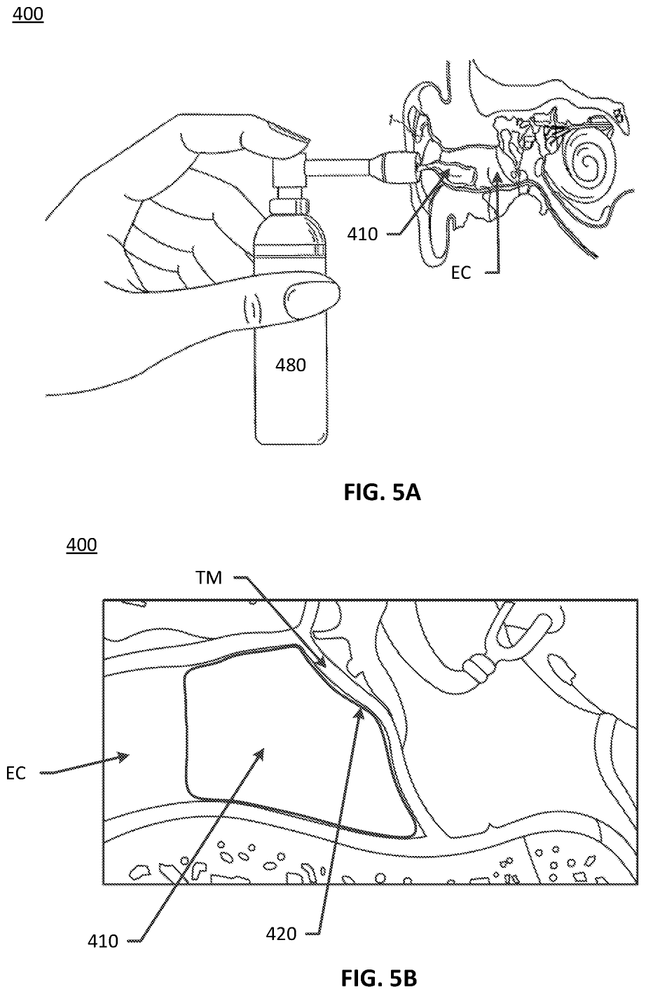

FIGS. 5A and 5B schematically illustrate a delivery system 400 according to another embodiment. In this embodiment, delivery system 400 includes a reservoir 410 disposed within ear canal EC. In this embodiment, reservoir 410 is formed in situ by injecting from a deployment device 480 a foamable carrier, or foam forming agent, mixed with therapeutic substance TS through the ear canal EC into the region adjacent tympanic membrane TM. The foam-based reservoir 410 enables delivery of therapeutic substance TS to tympanic membrane TM as the foam evaporates over time. The foam may also dissipate without leaving any residue, stains, or odor. The foam also provides a relatively uniform concentration of therapeutic substance TS at the surface of reservoir 410, such as at the delivery interface 420. The foam (or mousse) can be a lightweight material in cellular form that is made by introducing gas bubbles into a liquid phase. The carrier, or foam forming agent, can include foam producing agents and compounds that are able to generate a foamable composition when admixed with a liquid or gel composition. The foamable composition can generate a foam within deployment device 480 or upon dispensing from deployment device 480.

Suitable carrier compositions and techniques for delivering the foam are disclosed in U.S. Pat. No. 8,030,362 to Eliat, entitled “Compositions for Treatment of Ear Disorders and Methods of Use Thereof,” and in U.S. Patent Application Publication No. 2015/0342965 to Lozinsky et al, entitled “Foamable Otic Pharmaceutical Compositions,” the disclosures of which is incorporated herein by reference. An exemplary, suitable formulation for a foamable composition can include: (a) an oil-in-water emulsion that includes (i) the therapeutic agent in an effective concentration or amount, for example lidocaine in a 4% concentration solution; (ii) water in an amount of, for example, 75% or more (w/w), (iii) mineral oil in an amount of, for example, 15% or less (w/w); (iv) a synthetic surfactant pharmaceutically acceptable for otic applications; (v) a foaming agent; and (vi) white petrolatum; and (b) a compressed propellant gas.

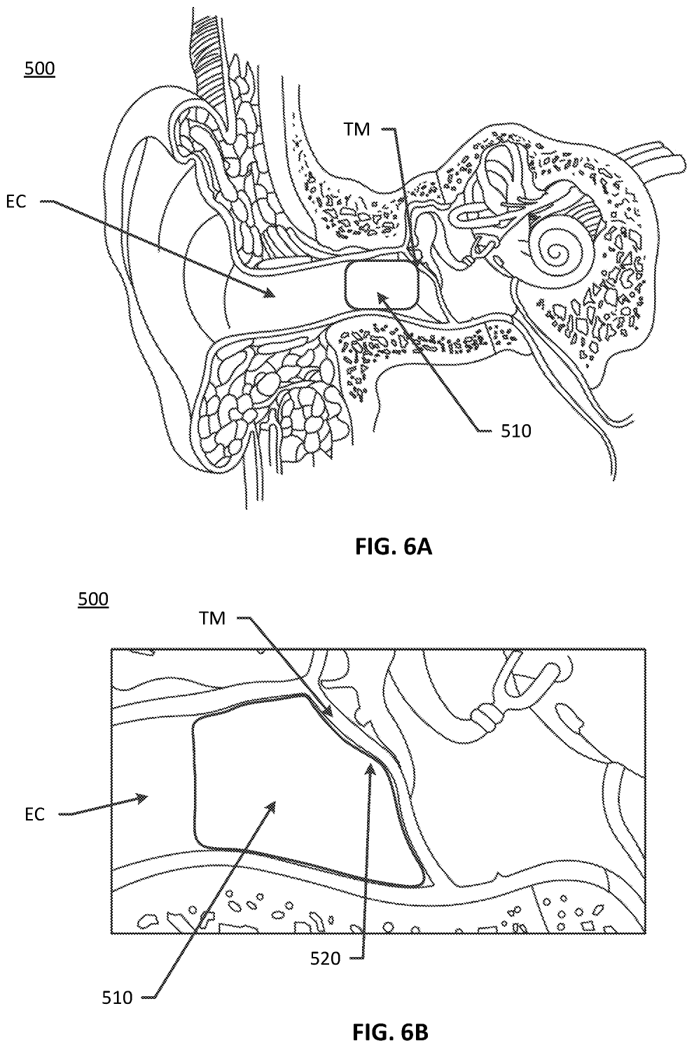

FIGS. 6A and 6B schematically illustrate a delivery system 500 according to another embodiment. Delivery system 500 is similar to delivery system 400, except that reservoir 510 can be formed in situ by injecting from a suitable deployment device (not shown) a hydrogel mixed with therapeutic substance TS through the ear canal EC into the region adjacent tympanic membrane TM. The hydrogel-based reservoir 510 enables delivery of therapeutic substance TS to tympanic membrane TM by osmotic transport.

A variety of hydrogel compositions may be suitable for delivery of different compositions of therapeutic substance TS. For example, photopolymerizable hydrogels disclosed in U.S. Pat. No. 5,410,016 to Hubbell et al, entitled “Photopolymerizable biodegradable hydrogels as tissue contacting materials and controlled-release carriers,” the disclosure of which is incorporated herein by reference may be used. The method of use may include mixing the therapeutic substance TS with an aqueous solution including a light-sensitive free-radical polymerization initiator and a macromer to form a coating mixture. The coating mixture can be applied to tympanic membrane TM (by any suitable means, such as a syringe) and then exposed to light sufficient to polymerize the macromer.

In other embodiments, the compositions and methods described in U.S. Pat. No. 6,201,065 to Pathak, et al., entitled “Multiblock biodegradable hydrogels for drug delivery and tissue treatment,” the disclosure of which is incorporated herein by reference, may be used. In these embodiments, macromers may be crosslinked reversibly or irreversibly to form gels for controlled delivery of therapeutic substance TS. The composition and properties of the macromers can be selected and fabricated to produce hydrogels with desired delivery properties (e.g., desired delivery rates or curves). The therapeutic substance TS may be provided in the macromer solution prior to or after administration, and either before or after gel formation, depending on the macromer composition. For example, the gels can be designed to have a selected rate of release of therapeutic substance TS, such as first order or zero order release kinetics. For specific therapeutic substances, such as peptides, the composition of the gel may be designed to result in pulsatile or mixed wave release characteristics in order to obtain maximum efficacy and to minimize side effects and tolerance development. The release profiles (e.g., delivery rates and changes in rate of delivery) can be selected by the use of macromers and gels formed therefrom that respond to specific external stimuli such as ultrasound, temperature, pH or electric current. For example, the extent of swelling and size of these hydrogels can be modulated. Changes induced in the swelling directly correlate to the rate of release of the incorporated therapeutic substances. Through this, a particular release profile may be obtained. The hydrogels may be degradable (e.g., biodegradable) so that removal is not required after administration or delivery. The gels permit controlled delivery and release of a biologically active therapeutic substance TS in a predictable and controlled manner locally at the tympanic membrane TM. Therefore, in use, an external stimulus can be applied to delivery system 500 such that the external stimulus modifies a rate of delivery of the therapeutic substance to the tympanic membrane TM. The external stimulus can include, for example, ultrasound, a temperature adjustment, a pH adjustment, and/or an electric current.

In other embodiments, rather than a hydrogel, the reservoir 510 may be implemented with any non-Newtonian fluid that can be mixed with therapeutic substance TS and delivered through the ear canal EC into the region adjacent tympanic membrane TM. That is, the material can flow under the shear forces produced by the delivery device, and then not flow under the lower shear forces imposed by gravity and normal movement of the subject (e.g., as a result of coughing, talking, swallowing, crying, yawning, or otherwise), and thus can be retained in appropriate apposition with the tympanic membrane TM and deliver therapeutic substance TS, e.g., by osmotic transport. Suitable materials may be shear thinning, i.e. apparent viscosity decreases with increased stress.

In other embodiments, rather than a hydrogel, the reservoir 510 may be implemented with a fluid that can be mixed with therapeutic substance TS and delivered through the ear canal EC into the region adjacent tympanic membrane TM and that can thicken, gel, or solidify in place. That is, the material can be delivered in liquid form (by any suitable device, such as a syringe, sprayer, etc.) and then be retained on tympanic membrane TM and deliver therapeutic substance TS, e.g., by osmotic transport. Suitable compositions can include those similar to compositions employed for “liquid bandages,” e.g., polymers dissolved in solvents, which form a thin film when the solvent evaporates. Suitable polymers can include water-soluble polymers such as polyvinylpyrrolidone, alcohol-soluble polymers such as ethyl cellulose, pyroxylin/nitrocellulose, or poly(methactcrylate-isobutene-monoisopropylmaleate, and hexamethyldisoloxane- or isooctane-soluble polymers such as acrylate or siloxane.

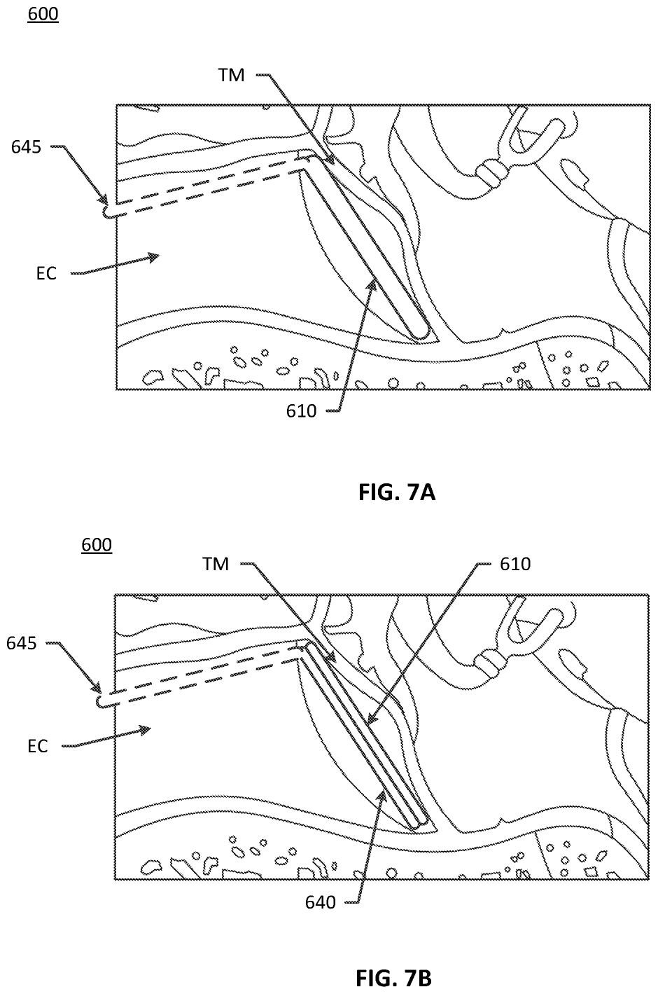

FIG. 7A schematically illustrates a delivery system 600 according to another embodiment. Delivery system 600 includes a reservoir 610 implemented as stable, solid, relatively thin film that may contain therapeutic substance TS, and that may be delivered through the ear canal EC and secured to tympanic membrane TM. Reservoir 610 can thus can be retained in appropriate apposition with the tympanic membrane TM and deliver therapeutic substance TS, e.g., by osmotic transport. Delivery system 600 may optionally include a retrieval element 645, such as a long tab that is secured at its distal end to reservoir 610 and that can extend to a proximal end near or outside of the entrance to the ear canal where it can be grasped by a user to retrieve reservoir 610 after the therapeutically effective amount of therapeutic substance TS has been delivered to tympanic membrane TM.

In another embodiment, shown in FIG. 7B, the stable, relatively thin film can be devoid of therapeutic substance, and instead form a body or substrate 640 supporting reservoir 610. In this embodiment reservoir 610 can be a thin layer of any suitable material, including those discussed above, that can contain therapeutic substance TS.

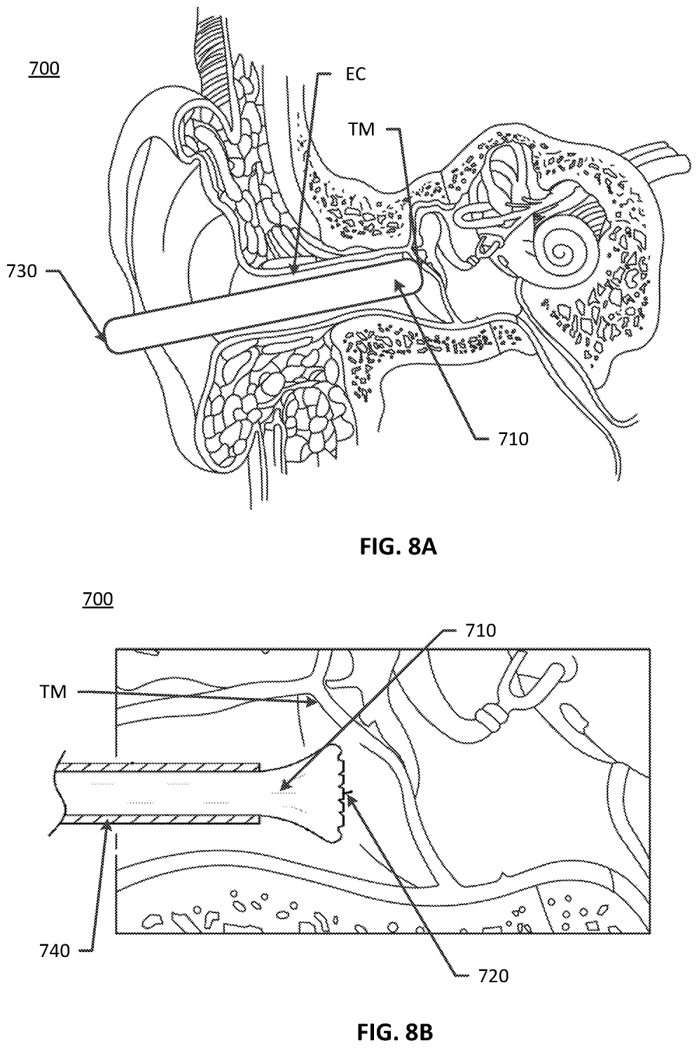

FIGS. 8A and 8B schematically illustrate a delivery system 700 according to another embodiment. In this embodiment, reservoir 710 is implemented as a bundle or other mass of wicking, fibrous material, i.e. a material that can absorb therapeutic substance TS in liquid form, and conduct therapeutic substance TS by capillary action through the body of reservoir 710 to a distal surface of reservoir 710, defining a delivery interface 720, in operative apposition with tympanic membrane TM, from which therapeutic substance TS can be delivered to tympanic membrane TM by osmotic transport. In some embodiments, as depicted in FIGS. 8A and 8B, a distal end of reservoir 710 (e.g., a portion of reservoir 710 including delivery interface 720), can have an outer diameter larger than an outer diameter of the proximal end of the reservoir 710. In some embodiments, reservoir 710 (e.g., the distal end of the reservoir) can expand after being disposed in the ear canal.

Reservoir 710 may include a body 740, such as a tube of material that radially constrains the fibrous material and increases its rigidity or stiffness to facilitate insertion through the ear canal EC. For example, as shown in FIG. 8B, a portion of reservoir 710 proximal of the distal end of reservoir 710 can be disposed within the body 740, and the body 740 can radially constrain the portion of reservoir 710 disposed within the body 740. A distal end of reservoir 710 can have an outer diameter larger than an outer diameter of the body 740 such that the outer diameter of the distal end of reservoir 710 is larger than an outer diameter of the portion of reservoir 710 disposed within body 740.

As needed, fluid including therapeutic substance TS can be added to reservoir 710 by supplying, or resupplying, it, e.g., in liquid form, to a proximal surface or end of the wicking material, serving as inlet 730. The proximal end of reservoir 710 can receive the fluid including therapeutic substance TS such that the fluid including therapeutic substance TS can be conducted to delivery interface 720 while delivery interface 720 is in operative apposition with tympanic membrane TM.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
disposing a delivery system including a delivery interface in an ear canal of an ear such that the delivery interface is in operative apposition with a tympanic membrane of the ear, the delivery system configured to change configuration after being disposed in the ear canal; and
maintaining the delivery interface in operative apposition with the tympanic membrane until a therapeutically effective dose of the therapeutic substance is delivered, via the delivery interface, to the tympanic membrane.

2. The method of claim 1, further comprising removing, after the maintaining, the delivery system from the ear.

3. The method of claim 2, wherein the delivery system includes a retrieval element, and the removing includes engaging the retrieval element and retracting the retrieval element in a proximal direction.

4. The method of claim 1, further comprising, supplying, after the disposing, the delivery system with the therapeutic substance by applying the therapeutic substance to a portion of the delivery system proximal of the delivery interface and spaced from the tympanic membrane.

5. The method of claim 1, wherein the delivery system is a degradable structure configured to degrade in response to contacting a tissue surface of the ear such that the therapeutic substance is released.

6. The method of claim 5, wherein the degradable structure is one of: a solid, a hydrogel, or a foam.

7. The method of claim 1, wherein the delivery system is a foam configured to dissolve in the ear canal without leaving a residue.

8. The method of claim 1, wherein the delivery system is a non-Newtonian fluid, and the disposing includes transporting, via a delivery device, the non-Newtonian fluid to a region of the ear canal adjacent to the tympanic membrane, the non-Newtonian fluid configured to flow under shear forces produced by the delivery device and to not flow under shear forces associated with gravity or a movement of the ear.

9. The method of claim 1, further comprising applying an external stimulus to the delivery system such that the external stimulus modifies a rate that the therapeutic substance is delivered to the tympanic membrane.

10. The method of claim 9, wherein the external stimulus includes at least one of: ultrasound, a temperature adjustment, a pH adjustment, and an electric current.

11. The method of claim 1, wherein the delivery system is a liquid when being disposed in the ear canal, the method further comprising altering, after the disposing, the liquid by at least one of thickening or solidifying the liquid.
